(19) United States
(12) Patent Application Publication
(10) Pub. No.: US 2022/0154197 A1
(43) Pub. Date: May 19, 2022

(54) CANNABINOID PRECURSOR PRODUCTION

(71) Applicant: PERPETUUM CROPSCIENCE BVBA, Antwerpen (BE)

(72) Inventors: Jeroen HOFENK, Antwerpen (BE); James TROCH, Antwerpen (BE)

(21) Appl. No.: 17/437,767

(22) PCT Filed: Mar. 11, 2020
(86) PCT No.: PCT/EP2020/056471

§ 371 Date: Sep. 9, 2021

(30) Foreign Application Priority Data
Mar. 12, 2019 (EP) 19162347.9

(51) Int. Cl. C12N 15/82; C07K 14/415; C12N 9/02

(52) U.S. Cl. CPC C12N 15/8243; C07K 14/415; C12Y 121/03008; C12Y 121/03007; C12N 9/0004

(57) ABSTRACT

A nucleic acid molecule is disclosed for transiently transforming a plant to produce Δ9-tetrahydrocannabinolic acid synthase, cannabidiolic acid synthase, and/or cannabichromenic acid synthase. The nucleic acid molecule corresponds to a nucleotide sequence comprising at least one of a nucleotide sequence fragment encoding a polypeptide having at least 78% sequence identity to SEQ ID NO: 4, SEQ ID NO:5 or SEQ ID NO:6 or comprising at least 15 contiguous nucleotides of the nucleotide sequence SEQ ID NO: 4, SEQ ID NO:5 or SEQ ID NO:6. The nucleotide sequence further comprises a KDEL or HDEL retrieval tag for targeting the nucleotide sequence to the endoplasmic reticulum. Further aspects relate to a viral vector comprising such nucleic acid molecule, and to methods for producing THCAS, CBDAS, CBCAS, THCA, CBDA, CBCA, THC, CBD and/or CBC based on transient expression of the nucleic acid sequence in a host plant.

### FIELD OF THE INVENTION

The present invention relates to the field of cannabinoid production. More specifically, the present invention relates to a process for producing cannabinoid precursors, such as 49-tetrahydrocannabinolic acid synthase (THCAS), cannabidiolic acid synthase (CBDAS), and/or cannabichromenic acid synthase (CBCAS), in transiently transformed plants, and to a construct that facilitates such transient expression.

### BACKGROUND OF THE INVENTION

Cannabinoids are a group of C21 compounds that, chemically, belong to the terpenophenols. Cannabinoids are produced naturally in humans (termed endocannabinoids) and by several plant species (termed “phytocannabinoids”), including Cannabis sativa. For example, they occur in resin produced by glandular hairs of C. sativa L. Among the over 420 known constituents of Cannabis, more than 60 belong to cannabinoids. Cannabinoids are accumulated in the glandular hairs, which account for more than 80% of the subcuticular secretion. Generally, these can be present in all plant parts, except the seeds. Cannabinoids bind to specific cannabinoid receptors and other target molecules to modulate a wide range of physiological processes such as neurotransmitter release.

In Cannabis plants, cannabinoids are produced by the metabolism of the plant in the form of carboxylic acids. However, a range of other types of cannabinoids have been detected in Cannabis. For a clear phytochemical discussion of the cannabinoids, they can, for convenience, be divided into three groups: acidic cannabinoids; neutral cannabinoids; and ‘artifacts’. This practical classification of the cannabinoids is illustrated in FIG. 1.

An important distinction that can be made within the group of cannabinoids is between the so-called acidic and neutral cannabinoids. Consequently, in fresh plant material almost no neutral cannabinoids can be found, but theoretically all cannabinoids are present in this acidic form. These can be converted into their decarboxylated analogues under the influence of light, heat, or prolonged storage, by losing the relatively unstable carboxylic group in the form of carbon dioxide.

The group of the acidic cannabinoids includes a large number of structures. The most common types of acidic cannabinoids found in a typical drug-type Cannabis plant are THCA, CBDA, CBGA, and CBCA. These acids can be converted to their neutral counterparts by decarboxylation to form THC, CBD, CBG, and cannabichromene (CBC), respectively. An example of this conversion is shown hereunder:

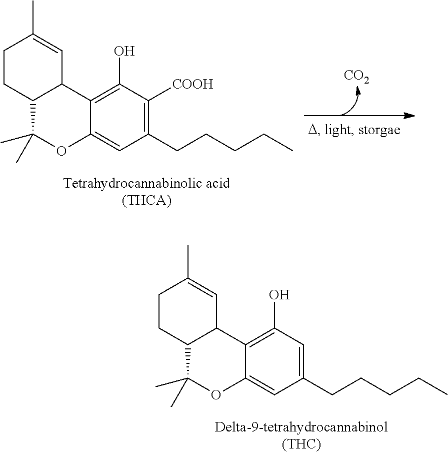

The group of cannabinoids that occur as a result of degradative conditions deserve some special attention, because their presence is largely the result of variable and unpredictable conditions during all the stages of growing, harvesting, processing, storage, and use. As a result, a well-defined Cannabis preparation may change rapidly into a product with significantly different biological effects. Degradation of THC results in the formation of CBN and delta-8-THC, whereas THCA can further degrade into CBNA.

Cannabinoids have been shown to have several beneficial medical/therapeutic effects, and therefore they are an active area of investigation for use in pharmaceutical products for various diseases and/or pain relief.

Currently the production of cannabinoids for pharmaceutical applications is done through chemical synthesis or through the extraction of cannabinoids from plants that are producing these cannabinoids, for example Cannabis sativa. However, there are several drawbacks to the current methods of cannabinoid production.

Synthetic cannabinoids comprise highly developed drugs designed by the pharmaceutical industry for therapeutic drug applications. The aim of such products is typically to achieve some benefit of natural Cannabis and translate it into a synthetic medicine that can be manufactured with consistency. This way, it is also easier to fulfill the legal requirements for prescription drugs, which typically demand a high purity and consistency in composition and concentrations. While whole-plant Cannabis is regularly used for its natural healing properties, it comes in so many varieties and variations that it can be challenging to push it through the regulatory processes that govern prescription drug approval in most countries. Synthetic medicines, on the other hand, can be easily replicated with consistency and high purity. The level of purity required by the pharmaceutical industry is reflected by the fact that no cannabinoid production process based on plant extracts has received approval yet from the U.S. Food and Drug Administration (FDA), while some synthetic compounds have been approved.

However, the chemical synthesis of various cannabinoids is a costly process when compared to the extraction of cannabinoids from naturally occurring plants. The chemical synthesis of cannabinoids can also involve the use of chemicals that are not environmentally friendly. The environmental effect can be considered as an additional cost to the production, adding to an already less desirable economical cost-effectiveness. Prior-art chemical synthesis approaches may rely on a complex, multi-step synthesis that leads to low yields and high production costs. Furthermore, various synthetic cannabinoids have been found to be less pharmacologically active as those extracted from plants such as Cannabis sativa. Therefore, a need exists in the art for alternatives to prior-art chemical synthesis approaches that are more cost-effective, environmentally-friendly and effective in pharmaceutical applications.

In contrast to the synthetic chemical production of cannabinoids, other methods are known to produce cannabinoids based on plants that naturally produce these chemicals. The most used plant for this purpose is Cannabis sativa. The plant Cannabis sativa is typically cultivated. During the flowering cycle, various cannabinoids are produced naturally by the plant. The plant can then be harvested for acquire the cannabinoids. The cannabinoids can be ingested directly from the plant itself for therapeutic purposes, or the cannabinoids can be extracted from the harvested plant material. There are various methods known in the art to extract the cannabinoids from the Cannabis sativa plant material. Such methods typically involve placing the plant material containing the cannabinoids in a chemical solution that selectively solubilizes the cannabinoids. Various suitable chemical solutions can be used, such as hexane, ethanol and butane. Cold water extraction methods and subcritical or supercritical CO₂ extraction methods are also known in the art. The chemical solution, thus containing the cannabinoids, can then be removed, leaving behind the excess plant material. The cannabinoid containing solution can then be further processed for use.

There are several drawbacks of the natural production and extraction of cannabinoids in plants, such as Cannabis sativa. Since there are numerous cannabinoids produced by Cannabis sativa it is often difficult to reproduce specific cannabinoid profiles in plants using an extraction process. Furthermore, variations in genotype and/or phenotype of the plant and environmental conditions can lead to variations in growth and may lead to different levels of cannabinoids in the plant material, thus making reproducible extraction difficult. It should be noted that, for pharmaceutical use, a consistent end product is typically required. Different cannabinoid profiles can have different pharmaceutical effects, which is not acceptable for a pharmaceutical product. Furthermore, the extraction of cannabinoids from Cannabis sativa extracts would appear to inevitably produce a mixture of cannabinoids, and not a highly pure single pharmaceutical compound. For example, since many cannabinoids are very similar in structure, it is difficult to purify these mixtures to a high level, e.g. resulting in cannabinoid contamination of the end product.

Other approaches for the production of cannabinoids are being actively investigated in biotechnological research. Compared to a cannabinoid production in plants, the production using a heterologous host system may have several potential advantages, such as a good process scalability enabling higher space-time yields, highly controllable and standardized processes, e.g. compliant with Good Manufacturing Practices (GMP), and supply management as well as a decreased risk of an illicit use or production. Furthermore, the enzymes THCAS, CBDAS and CBCAS use the same substrate for conversion. The establishment of a chassis strain able to produce CBGA would therefore allow for a tailored production of different cannabinoids or cannabinoid compositions depending on which genes of these cannabinoid producing enzymes are expressed in the chassis strain. Many companies are currently attempting to produce cannabinoids in yeast, e.g. using proprietary approaches. Such approaches may typically be at least conceptually based on, or inspired by, the publication by Luo et al, “Complete biosynthesis of cannabinoids and their unnatural analogues in yeast,” Nature 567, pp. 123-126 (https://doi.org/10.1038/s41586-019-0978-9).

In this approach by Xiaozhou Luo, Jay Keasling and their colleagues, a series of genetic changes are introduced into the yeast Saccharomyces cerevisiae. By tweaking yeast genes, and inserting others from bacteria and the Cannabis plant, the team created an organism capable of carrying out all the chemical reactions that are involved in cannabinoid production. Because the enzymes in the cannabinoid pathway are “a little sloppy”, as Keasling puts it, the team could also introduce fatty acids that the yeast would incorporate into cannabinoids, to obtain spawned variants of THC and CBD that are not found in nature. However, at the reported yields, this platform is not yet attractive for commercialization. It remains desirable to dramatically improve the yeast's efficiency and the fermentation protocol to obtain a biosynthetic approach that is cost-competitive with plant-extracted cannabinoids.

Transforming yeast into cannabinoid microfactories still poses considerable challenges. Although the protocol disclosed in the publication mentioned hereinabove involves 16 genetic modifications, the overall efficiency of the procedure is constrained by a single bottleneck: an enzyme that is needed for CBG production. Researchers have characterized the enzyme, known as a prenyltransferase, around a decade ago in a strain of medical Cannabis. However, using this Cannabis-derived enzyme in yeast proved unsuccessful, i.e. the yeast did not produce CBG. An alternative to prenyltransferase was found that is encoded in another variety of Cannabis. This alternative, when introduced into the yeast, enabled the researchers to produce CBG and its derivatives. However, the yields remain low, such that this process, that still requires 16 genetic modifications, is, as such, likely not economically feasible.

### SUMMARY OF THE INVENTION

It is an object of embodiments of the present invention to provide efficient, reproducible, safe, simple, environment-friendly, and/or cost-effective means and methods for, or relating to, the production of cannabinoid precursor enzymes in transiently transformed plants, such as Nicotiana benthamiana plants.

It is an advantage of embodiments of the present invention that cannabinoid precursor enzymes can be produced from biological material in a reproducible and/or constant manner and/or on an industrial scale, e.g. in an easily scalable process and/or with sufficiently high yields.

It is an advantage of embodiments of the present invention that a cost-effective, environmentally friendly, and/or more efficient alternative to traditional field- or greenhouse cultivation and/or chemical synthesis on industrial scale of commercially valuable cannabinoid precursor enzymes is provided.

It is an advantage of embodiments of the present invention that a high production effectiveness can be achieved for producing cannabinoid precursor enzymes without the limiting factors of cultivation times in traditional field- or greenhouse cultivation of Cannabis. For example, a transiently transformed Nicotiana benthamiana plant can produce cannabinoid precursor enzymes in 5 weeks of growing the plant and an additional 7 days post infiltration, versus 90 to 135 days with traditional cultivation of Cannabis (90 days for autoflowering species). This confers to about 3 times the speed to produce THC(a), CBD(a) and/or CBC(a). Furthermore, the THC(a), CBD(a) and/or CBC(a) may be used as a basis to produce other cannabinoids by transformation via degradation or isomerization.

It is an advantage of embodiments of the present invention that one or more disadvantages of a prior art method for producing cannabinoid precursor enzymes, such as methods and their associated disadvantages that were briefly outlined hereinabove, may be overcome or alleviated.

It is an advantage of embodiments of the present invention that a cheap and easily obtainable plant species, e.g. wild-type Nicotiana benthamiana, can be used as platform to obtain said cannabinoid precursor(s), for which suitable and efficient cultivation methods are well-known. Such plants can be used for economically feasible and faster large scale production of enzymes involved in cannabinoid biosynthesis.

It is an advantage of embodiments of the present invention that the modularization of THCAS, CBDAS, and CBCAS encoding catalysts to Nicotiana benthamiana species by ER (Endoplasmic Reticulum) localization can be used.

It is an advantage of embodiments of the present invention that sugar adornments that limits to the activity of enzymes crucial to the cannabinoid pathway can be reduced or avoided, e.g. such as the attachment of sugars to proteins in yeast, which could lead to lower yields.

It is an advantage of embodiments of the present invention that toxicity of cannabinoids to the host species can be reduced or avoided. For example, organisms such as yeast or E. coli may suffer from a toxicity of cannabinoids. It may be assumed that, typically, such molecules may have evolved in plants as a defense mechanism against insects, microorganisms and other biological threats. This means that such chemicals can often be deadly to the organisms when engineered to manufacture them. By using a similar plant species, such problems can be reduced or even avoided.

A method, viral vector, and/or nucleic acid molecule in accordance with embodiments of the present invention achieves the above objective.

In a first aspect, the present invention relates to a nucleic acid molecule, e.g. an isolated nucleic acid molecule, for transiently transforming a plant to produce 49-tetrahydrocannabinolic acid synthase (THCAS), and/or cannabidiolic acid synthase (CBDAS), and/or cannabichromenic acid synthase (CBCAS). The nucleic acid molecule corresponds to a nucleotide sequence, e.g. a nucleotide sequence as described hereinbelow. “Corresponding to” may refer to the nucleic acid molecule being encoded (e.g. directly) by the nucleotide sequence or a straightforward equivalent thereof, such as a nucleic acid molecule being encoded by a codon degenerate equivalent of the nucleotide sequence, and/or being encoded by a reverse and/or complement of the nucleotide sequence, and/or being encoded by a reverse and/or complement of a codon degenerate equivalent of the nucleotide sequence, and/or a homolog (homologous coding sequence) thereof.

The nucleotide sequence comprises at least one of following: (e.g. as a catalytic nucleotide sequence fragment)
i) a nucleotide sequence fragment encoding a polypeptide (which may have THCAS activity) having at least, e.g. greater than, e.g. about, 78%, preferably at least 82%, preferably at least 96%, e.g. at least 98%, e.g. at least 99%, e.g. 100% or complete, sequence identity to SEQ ID NO: 4 (or to the reverse and/or complement of SEQ ID NO: 4);
ii) a nucleotide sequence fragment comprising at least (or about) 15 contiguous nucleotides of the nucleotide sequence SEQ ID NO: 4 (or of the reverse and/or complement of SEQ ID NO: 4);
iii) a nucleotide sequence fragment encoding a polypeptide (which may have CBDAS activity) having at least, e.g. greater than, e.g. about, 78%, preferably at least 82%, preferably at least 96%, e.g. at least 98%, e.g. at least 99%, e.g. 100% or complete, sequence identity to SEQ ID NO: 5 (or to the reverse and/or complement of SEQ ID NO:);
iv) a nucleotide sequence fragment comprising at least (or about) 15 contiguous nucleotides of the nucleotide sequence SEQ ID NO: 5 (or of the reverse and/or complement of SEQ ID NO: 5);
v) a nucleotide sequence fragment encoding a polypeptide (having CBCAS activity) having at least, e.g. greater than, e.g. about, 78%, preferably at least 82%, preferably at least 96%, e.g. at least 98%, e.g. at least 99%, e.g. 100% or complete, sequence identity to SEQ ID NO: 6 (or to the reverse and/or complement of SEQ ID NO: 6); and;
vi) a nucleotide sequence fragment comprising at least (or about) 15 contiguous nucleotides of the nucleotide sequence SEQ ID NO: 6 (or of the reverse and/or complement of SEQ ID NO: 6).

The term “fragment” does not necessary mean that the intended sequence part is only part of a larger sequence, e.g. the fragment may refer to the whole sequence SEQ ID NO:4, SEQ ID NO:5 and/or SEQ ID NO:6, or may even comprise more than (any of) said sequences.

The nucleotide sequence further comprises a KDEL or HDEL retrieval tag, e.g. such that the KDEL or HDEL retrieval tag is being transcribed to the C-terminus of a protein encoded by the nucleotide sequence, for targeting the nucleotide sequence to the endoplasmic reticulum. For example, such KDEL retrieval tag may code for the target peptide sequence Lys-Asp-Glu-Leu, as known in the art. For example, such HDEL retrieval tag may code for the target peptide sequence His-Asp-Glu-Leu, as known in the art.

A nucleic acid molecule in accordance with embodiments of the present invention may further comprise a polyhistidine (poly-his) tag, such as a tag coding for a sequence of at least 2, e.g. at least 6, e.g. 6, e.g. at least 8 histidines, or other purification tag to facilitate purification. The purification tag may be placed in the nucleic acid sequence of the nucleic acid molecules codes for a poly-his or other purification tag known in the art at the C-terminus of the corresponding protein.

A nucleic acid molecule in accordance with embodiments of the present invention may be coupled to, e.g. may comprise, at least one heterologous moieties and/or at least one linker and/or at least one signal sequence and/or at least one detection label (or may comprise a nucleotide sequence fragment coding for any one or more of said features).

For example, the nucleic acid molecule may comprise at least one signal sequence for a corresponding host plant species, e.g. pr1a of tobacco (e.g. Nicotiana benthamiana or Nicotiana tabacum). A signal sequence for another host plant species, such as Arabidopsis thaliana, Hordeum vulgare, Oryza sativa, Solanum tuberosum and/or other plants, may be used. For example, the signal sequence may comprise a Nicotiana tabacum PR-1a signal peptide coding sequence (see e.g. UniprotKB id Q40557, entry version 66, sequence version 1, last sequence update Nov. 1, 1996; e.g. only the signal peptide part thereof: positions 1-30 of sequence), for example added to the N-terminus. Other examples of such signal sequences include:
for Oryza sativa: MASSSSRLSC CLLVLAAAAM AATA (UniprotKB accession id A0N0C1, TrEMBL sequence version 1, entry date: 2020-02-26, sequence date 2006-12-12);
for Arabidopsis thaliana: MKIFNSSQNL FLAITFFLVL IVHLKA (UniprotKB Q39188, TrEMBL sequence version 1, entry date: 2020-02-26, sequence date 1996-11-01);
for Hordeum vulgare: Pathogenesis-related protein 4—MAARLMLVAA LLCAATAMAT A (UniprotKB P93180, TrEMBL sequence version 1, entry date: 2019-12-11, sequence date 1997-05-01);
for Solanum tuberosum: Pathogenesis-related protein STH-2—MGVTSYTHET TTPIAPTRLF KALVV (UniprotKB P17642, Swiss-Prot sequence version 1, entry date 2020-02-26, sequence date 1990-08-01)

A nucleic acid molecule in accordance with embodiments of the present invention may comprise or correspond to (or may be a straightforward equivalent thereof) the nucleotide sequence SEQ ID NO: 1, or SEQ ID NO:2, or SEQ ID NO:3, or any combination thereof.

In a second aspect, the present invention relates to a viral vector comprising a nucleic acid molecule in accordance with embodiments of the first aspect of the present invention.

A viral vector in accordance with embodiments of the present invention may comprise a further nucleotide sequence for deglycosylation, e.g. a bacterial PNGase F gene sequence (see e.g. UniprotKB id P21163, entry version 107, sequence version 2, last sequence update Nov. 1 1991).

In a viral vector in accordance with embodiments of the present invention, the nucleotide sequence and/or the further nucleotide sequence may be codon-optimized for Nicotiana benthamiana or Nicotiana tabacum species. The further nucleotide sequence and the nucleotide sequence may be co-integrated in a single sequence.

However, other host plants are not necessarily excluded. For example, (and optionally) the nucleotide sequence and/of further nucleotide sequence may be codon-adoption index optimized for different species, as will be understood by the skilled person. Other illustrative plant species may include: Arabidopsis thaliana, Hordeum vulgare, Oryza sativa, Solanum tuberosum and/or other plants, preferably which can be easily grown and/or cultivated.

In a third aspect, the present invention relates to a method for producing 49-tetrahydrocannabinolic acid synthase (THCAS), and/or cannabidiolic acid synthase (CBDAS), and/or cannabichromenic acid synthase (CBCAS). The method comprises transiently transforming a plant with a nucleic acid molecule in accordance with embodiments of the first aspect of the present invention. The method comprises extracting THCAS and/or CBDAS and/or CBCAS from plant biomass obtained from the transiently transformed plant.

The nucleic acid molecule a HDEL, or preferably a KDEL, retrieval tag, to redirect the catalytic nucleotide sequence fragment(s) i), ii), iii), iv), v) and/or vi) to the endoplasmic reticulum (ER) of the host plant, e.g. a Nicotiana benthamiana or Nicotiana tabacum species plant. Thus, the method can enable ER/apoplast targeting to obtain active THCAS and/or CBDAS and/or CBCAS. Post-translational modifications, such as glycosylation obtained in the ER, could contribute to the correct folding of the enzyme, since deglycosylation of the protein has at least no negative effect on the activity and therefore on the stability of the native enzyme.

In a method in accordance with embodiments of the present invention, the plant may be a Nicotiana benthamiana or Nicotiana tabacum plant. The plant (expression host) may be any wild-type Nicotiana benthamiana cultivar. The use of transgenic Nicotiana benthamiana or Nicotiana tabacum related species which are permanently transformed, expressing TCHAS, CBDAS, or CBCAS genes are also within the scope of the present invention.

It is an advantage that a method in accordance with embodiments of the present invention may reduce production time significantly, e.g. by as much as 70%, versus traditional extraction and/or purification of THCA, CBDA and/or CBCA from Cannabis biomass.

A method in accordance with embodiments of the present invention may comprise filtrating and/or purifying the extracted THCAS and/or CBDAS and/or CBCAS, e.g. by a chromatography process.

In a method in accordance with embodiments of the present invention, the chromatography process may comprise immobilized metal affinity chelating chromatography.

In a method in accordance with embodiments of the present invention, the plant may also, e.g. simultaneously, be transiently transformed to (co-)express a deglycosylation sequence for obtaining the expression of THCA and/or CBDAS and/or CBCAS without glycosylation, e.g. to ensure the stability of the protein of interest.

A method in accordance with embodiments of the present invention may comprise introducing the nucleotide sequence, using the viral vector in accordance with embodiments of the second aspect of the present invention, into at least one Agrobacterium tumefaciens strain.

A method in accordance with embodiments of the present invention may comprise exposing, e.g. infecting, the plant with said at least one Agrobacterium tumefaciens strain.

In a method in accordance with embodiments of the present invention, the at least one Agrobacterium tumefaciens strain may comprise a combination of a plurality of (e.g. high yielding) Agrobacterium tumefaciens strains comprising or consisting of GV3101, C58C1, and LBA4404 and wild-type strains A4, At06, At10, and At77, to improve transfection rates.

In a fourth aspect, the present invention relates to a method for producing 49-tetrahydrocannabinolic acid (THCA), and/or cannabidiolic acid (CBDA), and/or cannabichromenic acid (CBCA), comprising a method in accordance with embodiments of the third aspect of the present invention.

The method comprises:
converting the (e.g. purified) THCAS to THCA through CBGA oxidocyclization without hydroxylation, e.g. by adding CBGA to the supernatant for 6 to 8 hours of incubation;
converting the (e.g. purified) CBDAS to CBDA through CBGA oxidocyclization without hydroxylation, e.g. by adding CBGA to the supernatant for 6 to 8 hours of incubation; and/or
converting the (e.g. purified) CBCAS to CBCA through CBGA oxidocyclization without hydroxylation, e.g. by adding CBGA to the supernatant for 6 to 8 hours of incubation.

The thus obtained THCA, CBDA and/or CBCA may be used, e.g. as is, for pharmaceutical applications in its acidic form.

The method may further comprise a decarboxylation performed on the obtained THCA, CBDA and/or CBCA to yield active THC, CBD and/or CBC.

The obtained THCA, CBDA and/or CBCA may be used as a basis for biosynthesizing other cannabinoids, e.g. by degradation and/or isomerization.

The independent and dependent claims describe specific and preferred features of the invention. Features of the dependent claims can be combined with features of the independent claims and with features of other dependent claims as deemed appropriate, and not necessarily only as explicitly stated in the claims.

The foregoing summary of the invention and following detailed description of the drawings and the detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 shows a classification of the cannabinoids and their production processes, to illustrate concepts relating to embodiments of the present invention.

FIG. 2 shows a vector map of a PR-1A/THCAS/PNGASE-F/ER/6×HIS construct, in accordance with embodiments of the present invention.

FIG. 3 shows a vector map of a PR-1A/CBDAS/PNGASE-F/ER/6×HIS construct, in accordance with embodiments of the present invention.

FIG. 4 shows a vector map of a PR-1A/CBCAS/PNGASE-F/ER/6×HIS construct, in accordance with embodiments of the present invention.

FIG. 5 shows a prediction of a signaling peptide sequence of CBCAS, for illustrating embodiments of the present invention.

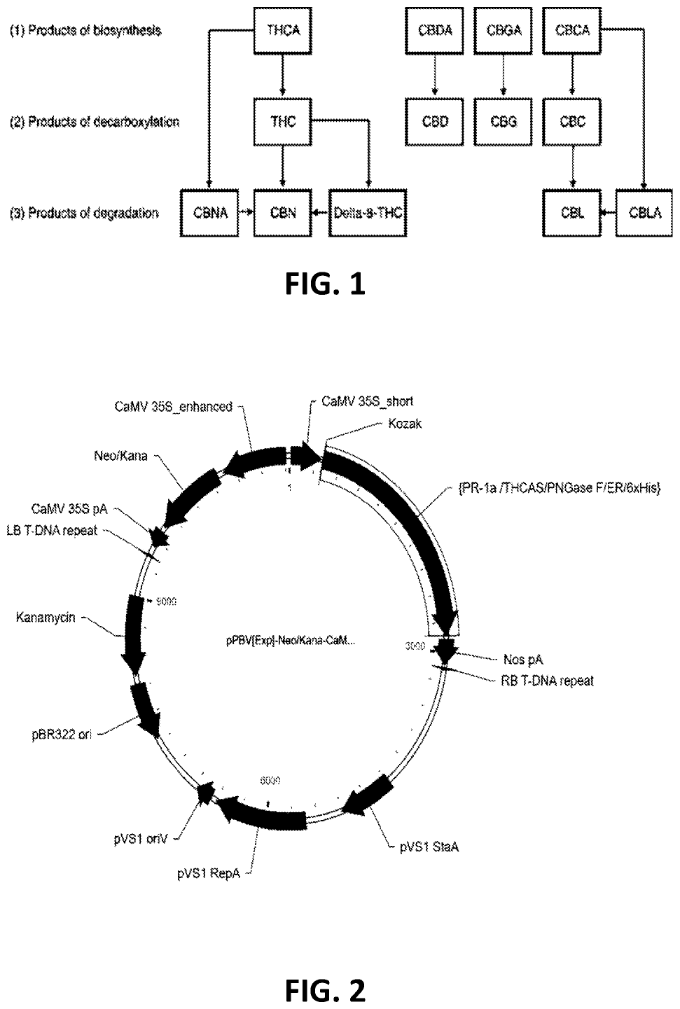

The drawings are schematic and not limiting. Elements in the drawings are not necessarily represented on scale. The present invention is not necessarily limited to the specific embodiments of the present invention as shown in the drawings.

### DETAILED DESCRIPTION OF EMBODIMENTS

Notwithstanding the exemplary embodiments described hereinbelow, is the present invention only limited by the attached claims. The attached claims are hereby explicitly incorporated in this detailed description, in which each claim, and each combination of claims as allowed for by the dependency structure defined by the claims, forms a separate embodiment of the present invention.

Raw materials being used are not always described in detail. Such raw materials may be commercially available products. While process steps and/or preparation methods are not always described in detail, such process steps and/or preparation methods may be considered to be well-known by those skilled in the art.

The word “comprise,” as used in the claims, is not limited to the features, elements or steps as described thereafter, and does not exclude additional features, elements or steps. This therefore specifies the presence of the mentioned features without excluding a further presence or addition of one or more features.

In this detailed description, various specific details are presented. Embodiments of the present invention can be carried out without these specific details. Furthermore, well-known features, elements and/or steps are not necessarily described in detail for the sake of clarity and conciseness of the present disclosure.

Embodiments of the present invention are specifically described below with reference to the embodiments, so as to facilitate the understanding of the present invention by those skilled in the art. It should be noted that the embodiments are only used for further explanation of the present invention and cannot be understood to necessarily limit the protection scope of the present invention. The person skilled in the art will recognize that the protection scope of the present invention can be better understood by those skilled in the art.

In a first aspect, the present invention relates to a nucleic acid molecule for transiently transforming a plant to produce Δ9-tetrahydrocannabinolic acid synthase (THCAS) and/or cannabidiolic acid synthase (CBDAS) and/or cannabichromenic acid synthase (CBCAS). The nucleic acid molecule corresponds to a nucleotide sequence comprising at least one of following:
i) a nucleotide sequence fragment encoding a polypeptide having at least 78% sequence identity to SEQ ID NO: 4 or comprising at least 15 contiguous nucleotides of the nucleotide sequence SEQ ID NO: 4; and/or
ii) a nucleotide sequence fragment encoding a polypeptide having at least 78% sequence identity to SEQ ID NO: 5 or comprising at least 15 contiguous nucleotides of the nucleotide sequence SEQ ID NO: 5; and/or
iii) a nucleotide sequence fragment encoding a polypeptide having at least 78% sequence identity to SEQ ID NO: 6 or comprising at least 15 contiguous nucleotides of the nucleotide sequence SEQ ID NO: 6.

The nucleotide sequence further comprises a KDEL or MEL retrieval tag for targeting the nucleotide sequence to the endoplasmic reticulum.

In a second aspect, the present invention relates to a viral vector comprising such nucleic acid molecule.

In a third aspect, the present invention relates to a method for producing Δ9-tetrahydrocannabinolic acid synthase (THCAS), and/or cannabidiolic acid synthase (CBDAS), and/or cannabichromenic acid synthase (CBCAS). The method comprises transiently transforming a plant with a nucleic acid molecule in accordance with embodiments of the first aspect of the present invention. The method comprises extracting THCAS and/or CBDAS and/or CBCAS from plant biomass obtained from the transiently transformed plant.

In a fourth aspect, the present invention relates to a method for producing Δ9-tetrahydrocannabinolic acid (THCA), and/or cannabidiolic acid (CBDA), and/or cannabichromenic acid (CBCA), comprising a method in accordance with embodiments of the third aspect of the present invention.

The method comprises:
converting the (e.g. purified) THCAS to THCA through CBGA oxidocyclization without hydroxylation, e.g. by adding CBGA to the supernatant for 6 to 8 hours of incubation;
converting the (e.g. purified) CBDAS to CBDA through CBGA oxidocyclization without hydroxylation, e.g. by adding CBGA to the supernatant for 6 to 8 hours of incubation; and/or
converting the (e.g. purified) CBCAS to CBCA through CBGA oxidocyclization without hydroxylation, e.g. by adding CBGA to the supernatant for 6 to 8 hours of incubation.

Thus, the present invention provides engineered recombinant THCAS, CBDAS, and/or CBCAS fusion constructs, e.g. the nucleic acid molecule referred to hereinabove, as well as viral vectors comprising such construct and methods involving the use thereof.

Embodiments may provide or enable a more efficient and cost-effective process, capable of producing enzymes that are involved in cannabinoid biosynthesis by transient transformation of a plant, e.g. Nicotiana benthamiana. An illustrative method in accordance with embodiments may comprise inserting nucleic acid molecule, e.g. incorporating the genes of interest, in Agrobacterium tumefaciens and introducing the construct by agroinfiltration into the endoplasmic reticulum (ER) of plant cells, e.g. of 5 weeks old Nicotiana benthamiana plants.

Nicotiana benthamiana may be considered as a bioreactor of choice for the transient expression of recombinant protein in a manufacturing setting. The small ornamental plant has a high leaf to stem ratio and is very prolific in hydroponic culture. Nicotiana benthamiana tolerates the transfection vectors and delivers maximum synthesis of heterologous proteins in 5-7 days after transfection. Scale-up of this bioreactor is a matter of growing more plants not re-engineering processes. However, the skilled person may transpose the findings of the present disclosure to other host plant species, as he deems suitable, which are therefore also considered to be covered by embodiments of the present invention.

Plants have advantageously all the required eukaryotic cell machinery to accurately produce plant, human and animal proteins. Thus, the bioreactor may be an individual plant. Plants are well suited to express complex proteins, and minimize risk by not supporting growth of human or animal pathogens.

A benefit of this approach is that the production of the cannabinoid is fast and continuous, low cost and reliable, and only a specific cannabinoid is produced or a subset is produced. The extraction and purification process of the cannabinoid may be straightforward since there is only a single cannabinoid or a selected few cannabinoids present in the plant biomass. The process can be upscaled in a linear fashion, e.g. simply by growing more plants. Furthermore, it is a sustainable process which is more environmentally friendly than synthetic production and can also be purified to meet the requirements for pharmaceutical applications.

The acidic forms of the cannabinoids (THCA, CBDA, and CBCA) obtained through CBGA oxidocyclization without hydroxylation of THCAS, CBDAS, and CBCAS respectively, may be used as a pharmaceutical product or the acidic cannabinoids can be turned into their neutral form for use, for example THC, CBD, and CBC may be produced from THCA, CBDA, and CBCA respectively through decarboxylation. The resulting cannabinoid products may be used in the pharmaceutical/nutraceutical industry, e.g. to treat a wide range of health issues.

The contacting can for example be achieved by mixing the CBGA with recombinant THCAS, CBDAS, and CBCAS in a solution and/or in an immobilized state under conditions and for a length of time suitable to convert at least about 10%, at least about 20%, at least about 30%, at least about 40%, at least about 50%, at least about 60%, at least about 70%, at least about 80%, at least about 90%, or at least about 100% of the CBGA to THCA, CBDA, and CBCA.

With regards to expressing the proteins in transiently transformed Nicotiana benthamiana, THCAS, CBDAS, and/or CBCAS encoding sequences may be fused to the endoplasmic reticulum (ER) retrieval tag, e.g. KDEL, and a poly-histidine tag, e.g. all fused to the C-terminus of the proteins (i.e. of the protein transcribed by the nucleic acid molecule). The KDEL tag allows proteins to accumulate in the ER, a strategy that may lead to higher accumulation and/or reduce in planta proteolytic degradation.

The THCAS construct comprises a catalytic nucleotide sequence (e.g. without the signal peptide) as represented by SEQ ID NO: 4, or a suitable analog or sufficient part thereof (i.e. sufficient to express the intended THCAS enzyme). Likewise, a CBDAS catalytic nucleotide sequence may be represented by SEQ ID NO: 5, and a CBCAS catalytic nucleotide sequence by SEQ ID NO: 6 (both considered with a tolerance for suitable analogs or substantial fragments thereof).

The nucleic acid molecule may comprise a bacterial PNGase F gene sequence for obtaining fully functional deglycosylated THCAS and/or CBDAS and/or CBCAS.

The nucleic acid molecule comprises an endoplasmic reticulum retrieval tag, e.g. a KDEL tag, e.g. located at the C-terminus of the protein.

The nucleic acid molecule may comprise a (nucleotide sequence fragment coding for a) signal peptide for the intended host plant, e.g. a Nicotiana tabacum PR-1a signal peptide.

The nucleic acid molecule may comprise a purification tag, such as a polyhistidine tag. For example, a tag peptide may be used, e.g. engineered into the primary structures of the engineered fusion enzyme, to facilitate purification of produced THCAS, CBDAS and/or CBCAS. Examples include a polyhistidine tag, a streptavidin (biotin-binding) tag, a flagellar antigen tag, a hemagglutinin tag, or a glutathionine S-transferase tag, among others.

For example, the nucleic acid molecule may comprise said catalytic nucleotide sequence for THCAS, CBDAS and/or CBCAS, a Nicotiana tabacum PR-1a signal peptide added to the N-terminus, followed by an endoplasmic reticulum (ER) retrieval tag KDEL and a polyhistidine tag, both fused to the C-terminus of the protein.

The nucleic acid molecule may be codon-optimized for expression in a specific host plant species, such as Nicotiana benthamiana species.

SEQ ID NO:1, SEQ ID NO:2 and SEQ ID NO:3 disclose nucleotide sequences of such nucleic acid molecules for respectively transiently expressing THCAS, CBDAS and CBCAS in Nicotiana Benthamiana, i.e. nucleotide sequences of respectively PR-1A/THCAS/PNGASE-F/ER/6×HIS, PR-1A/CBDAS/PNGASE-F/ER/6×HIS and PR-1A/CBCAS/PNGASE-F/ER/6×HIS constructs, each codon-optimized for Nicotiana benthamiana.

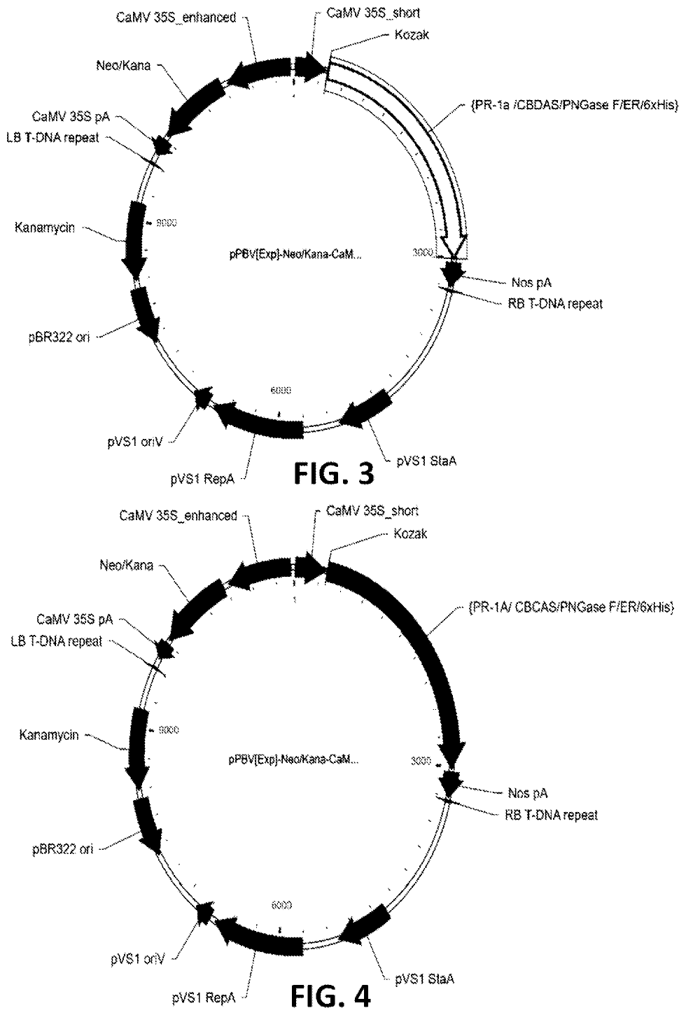

FIG. 2, FIG. 3 and FIG. 4 show corresponding viral vector maps of a viral vector in accordance with embodiments, comprising a nucleic acid molecule in accordance with embodiments for respectively transiently expressing THCAS, CBDAS and CBCAS in Nicotiana benthamiana.

The above constructs can also be cloned into two separate vectors; one functioning as the construct containing the PNGase F gene sequence, the other functioning as the construct containing the THCAS, CBDAS and/or CBCAS gene. Embodiments of the present invention may relate to a combination of such separate vectors.

A short (346 bp) but strong constitutive cauliflower mosaic virus 35S promoter (P35S), a kozak translation initiation sequence, and a nopaline synthase polyadenylation terminator signal for regulation of gene expression may be utilized. The use of constructs for N- and C-terminally truncated and both N- and C-terminally truncated versions of both nucleotide sequences and all homologous coding sequences, with a minimum of 45% sequence identity, are also within the scope of this present invention.

Embodiments of the present invention may relate to a composition comprising the nucleic acid molecule, e.g. with no additional protein components are present in said composition.

For example, the percentage by weight of the recombinant THCAS, CBCAS and/or CBDAS fusion enzyme (i.e. the nucleic acid molecule) in such composition may be from about 0.00001% to 99.99999%, e.g. from about 0.00001% to 99.99999%, e.g. from about 0.0001% to 99.9999%, e.g. from about 0.001% to 99.999%, e.g. from about 0.001% to 99.999%, e.g. from about 0.01% to 99.99%, e.g. from about 0.1% to 99.9%, e.g. from about 1% to 99%.

The acquired THCAS, CBDAS, and CBCAS after expression and purification can be further biosynthesized by CBGA through oxidocyclization without hydroxylation to obtain THCA, CBDA, and CBCA respectively. The enzymaticaly synthesized THCA, CBDA, and CBCA can then be carboxylated, e.g. by heating at 120° C. to obtain THC, CBD, and CBC respectively. The obtained THCA, CBDA, and CBCA can also function as a basis for further modification to other cannabinoids for example via degradation or isomerization.

In the agroinfiltration experiments discussed hereinabove, 5- to 7-weeks-old N. benthamiana plants were used. Nicotiana benthamiana seeds were grown in a greenhouse. Seedling and germination of Nicotiana benthamiana plants were carried out under light emitting diode (LED) illumination 24 hours/day, 7 days/week. Red and blue diodes were selected that match the action spectrum of photosynthesis (25% blue and 75% red). Other wavelengths may be less or not productive. The LEDs were focused on the plants. Plants were grown to usable maturity 20% faster by this approach as compared to other commercial solutions. All seeds were germinated using identical soil and fertilizer at 26.6° C.

For the biosynthesis of the genes of interest, respectively THCAS (UniProtKB-Q8GTB6, entry version 71, sequence version 1, last sequence update Mar. 1, 2003), CBDAS (UniProtKB-A6P6V9, entry version 50, sequence version 1, last sequence update Aug. 21, 2007), and CBCAS (GenBank: LY658672.1, cf. KR 1020190025485-A/8 11 Mar. 2019) from Cannabis sativa (hemp, marijuana), were used in combination with the bacterial PNGase F gene sequence (UniprotKB-P21163, entry version 107, sequence version 2, last sequence update Nov. 1 1991), a Nicotiana tabacum PR-1a signal peptide (UniprotKB-Q40557, entry version 66, sequence version 1, last sequence update Nov. 1, 1996; only signal peptide part: positions 1-30 of sequence) added to the N-terminus, a KDEL endoplasmic retrieval tag, and a poly-histidine tag added to the C-terminus, forming a template to biosynthesize the novel engineered recombinant THCAS, CBDAS, and CBCAS enzymes. The restriction sites for EcoRI and BgIII were added to the 5′ and 3′ ends of the gene, respectively. Codon usage was optimized for Nicotiana benthamiana expression, and the gene synthesis was done by Genscript Inc. The THCAS 2436 bp-fragment and CBDAS 2613 bp-fragment were cloned into pUC57 vectors to facilitate gene subcloning into plant expression vector. (See SEQ ID NO: 1, SEQ ID NO: 2 and SEQ ID NO: 3 for the full nucleotide sequences of these illustrative THCAS, CBDAS, and CBCAS constructs respectively, and FIGS. 2, 3, and 4 for their respective corresponding viral vector maps). N-linked glycosylation is a post-translational modification which is useful to correct folding, stability and biological activity of many proteins, including recombinant subunit vaccines and therapeutic proteins produced in heterologous expression systems. Some eukaryotic (as well as bacterial) proteins may not contain N-glycans in the native host, but their proteins may contain multiple potential glycosylation sites that are aberrantly glycosylated when these proteins are expressed in heterologous eukaryotic expression systems, potentially leading to impaired functional activity. Indeed, the attachment of carbohydrates may strongly affect the physico-chemical properties of a protein, therefore can alter its essential biological properties such as specific activity, ligand-receptor interactions and immunogenicity and may pose a safety risk when used in vivo. As THCAS, CBDAS, and CBCAS would appear to be glycosylated after expression and purification, it could be suggested that this modification would have an influence on the stability of the proteins. Thus, for producing deglycosylated proteins in plant cells, we transiently co-express bacterial PNGase F (Peptide: N-glycosidase F) in combination with the target protein of interest (THCAS, CBDAS and/or CBCAS). PNGase F is a 34.8-kDa enzyme secreted by a Gram-negative bacterium Flavobacterium meningosepticum. It cleaves a bond between the innermost GlcNAc and asparagine residues of high-mannose, hybrid and complex oligosaccharides in N-linked glycoproteins, except when the a (1-3) core is fucosylated.

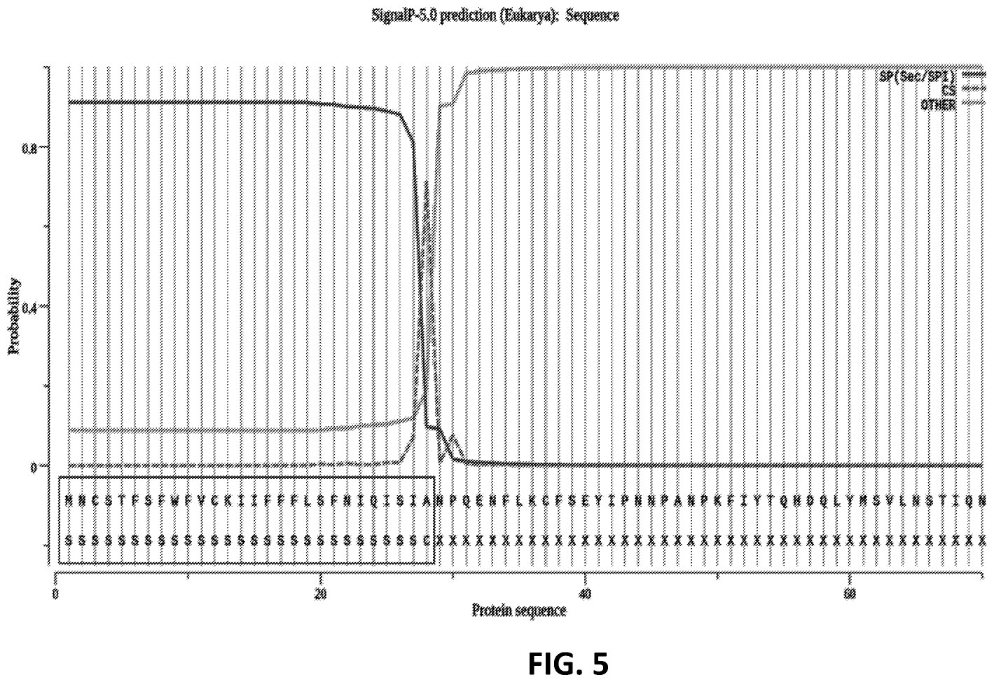

The Cannabis genome sequence was analyzed for genes with high similarity to THCA synthase using BLAST analysis. This led to the identification of a gene with 96% nucleotide similarity to THCA synthase. Based on subsequent biochemical characterization, the authors named this gene Cannabis sativa cannabichromenic acid synthase (CBCAS) and was deposited as the entire sequence, including signaling peptide sequence, in the Genbank database under registration: LY658672.1. We've predicted the signaling peptide sequence (see FIG. 5) and discarded these replicates.

As the native THCAS, CBDAS, and CBCAS genes employs tandem rare codons, which could reduce the efficiency of translation or even disengage the translational machinery, the codon usage bias in Nicotiana benthamiana was used by upgrading the codon adoption index (CAI) from 0.72 to 0.86. The GC content and unfavorable peaks have been optimized to prolong the half-life of the mRNA. The Stem-Loop structures, which impact ribosomal binding and stability of mRNA, were broken. In addition, negative cis-acting sites were screened and successfully modified.

For the construction of a plant expression vector, the entire THCAS 2436 bp-fragmental-orf, CBDAS 2613 bp-fragmental-orf, and CBCAS 2616 bp-fragmental-orf were excised from pUC57-THCAS, pUC57-CBDAS, and pUC57-CBCAS by digesting with both EcoRI and BglII and subcloned into pPRP[Exp]-CaMV35S binary vectors in the corresponding sites and under the control of 35S-promoter. The transformed colonies were confirmed by restriction digestion. The recombinant plasmids pCambia-THCAS, pCambia-CBDAS, and pCambia-CBCAS, were extracted from the selected colony and consequently was transformed into Agrobacterium to carry out agroinfilteration experiments.

The pPRP[Exp]-CaMV35S-THCAS, pPRP[Exp]-CaMV35S-CBDAS, and pPRP[Exp]-CaMV35S-CBCAS constructs were transformed into Agrobacterium tumefaciens strains GV3101 C58C1 and LBA4404 and wild-type strains A4, At06, At10, and At77 using electroporation technique at 2.5 kV, 25 mF and 400Ω. The transformed cells were plated on LB agar medium containing 50 mg/ml Ampicillin (Sigma Aldrich).

Agroinfiltration was used for transient expression in Nicotiana benthamiana with Agrobacterium tumefaciens strains as previously described. 100 μl of transformed Agrobacterium frozen cells stock was inoculated in 5 ml LB broth (Thermo Fisher Scientific) and supplemented with 50 mg/ml Ampicillin for THCAS, CBDAS, and CBCAS respectively. Overnight, the cultures were incubated at 28° C., shaking at 220 rmp. 2×500 μl was used to inoculate 2× 50 ml of LB medium. The cultural cells were incubated at 28° C. shaking at 220 rpm until the culture had reached an O.D.600=0.6. The cells were harvested by centrifugation at 6000 rpm and resuspended in 2×50 ml IVIES buffer (10 mM IVIES; pH 5.5, 10 mM MgCl2). These mixtures were incubated for 2.5 hours at room temperature with 120 μM acetosyringone and was added to the Agrobacterium suspension in infiltration buffer (1×MS, 10 mM MES, 2.5% glucose) for THCAS, CBDAS, and CBCAS respectively. For the effect of monosaccharide on induction of vir gene, different percentages of glucose (0, 1, 2 or 4%) were added to the Agrobacterium suspension in the infiltration buffer (lx MS, 10 mM IVIES, 200 μM acetosyringone). 5- to 7-weeks old N. benthamiana plants were infiltrated in a vacuum chamber by submerging N. benthamiana plant aerial tissues in Agrobacterium suspension and applying a 50-400 mbar vacuum for 30, 45 or 60 seconds.

The most optimal infiltration was routinely applied at 50-100 mbar for 60 sec. Once the vacuum was broken, infiltrated N. benthamiana plants were removed from the vacuum chamber, thoroughly rinsed in water, and grown for 5-7 days under the same growth conditions used for pre-infiltration growth. To avoid any variability, the leaves and location on the leaf, comparably-sized leaves for each plant of similar age were agro-infiltrated for each experiment.

For the Southern Blot Analysis, individual agroinfiltrated leaves with the pPRP[Exp]-CaMV35S-THCAS, pPRP[Exp]-CaMV35S-CBDAS, and pPRP[Exp]-CaMV35S-CBCAS constructs were harvested at different time-intervals; 4, 6, 8 and 10 days post-infiltration, in addition to un-infiltrated plants were used as control. DNA of infiltrated leaves was extracted by DNeasy Plant DNA mini kit (QIAGEN) and fragmented by endonuclease enzyme; EcoRI. Both recombinant THCAS 2436 bp-fragmental-orf, CBDAS 2613 bp-fragmental-orf, and CBCAS 2616 bp-fragmental-orf released from pUC57-THCAS, pUC57-CBDAS, and pUC57-CBCAS respectively were used as a probe. Labeling and detection were carried out using Biotin Deca Label DNA Labeling Kit (Thermo Fisher Scientific) and Biotin chromogenic Detection kit (Thermo Fisher Scientific) respectively.

For the Western Blot Analysis, individual agroinfiltrated Nicotiana bentamiana leaves were harvested and grinded in liquid nitrogen. Total proteins were extracted using SDS-extraction buffer (2% SDS, 0.2% bromopheol blue, 10% glycerol), and the extracts were clarified by centrifugation at 14,500 g for 20 min at 4° C. The, supernatants were transferred to fresh tubes, and the protein content of THCAS, CBDAS, and CBCAS were determined (Bradford assay—1976). Total proteins (40 μg each) were separated by SDS-PAGE and then transferred onto polyvinylidene difluoride (PVDF) membranes. Polyvinylidene difluoride membranes were blocked for at least 2 hours and then probed with rabbit anti-THCAS, rabbit anti-CBDAS, and rabbit anti-CBCAS in a 1:500 dilution. After extensive washing, the membrane was incubated with the appropriate secondary antibody in a 1:5000 dilution and then was conjugated to alkaline phosphatase. BCIP/NBT (Amresco) was used for immunodetection.

For the detection of chimeric gene by real-time polymerase chain reaction (RT-PCR) an illustra RNAspin mini kit (GE healthcare) was used to extract, total RNA from the agroinfiltrated leaves. Oligonucleotides pair at the core region was designed to detect the presence of the THCAS, CBDAS, and CBCAS genes at the core region; using THCAS-specific forward primer: 5′-CTCGTATACACTCAACACGACC-3 ‘ (SEQ ID NO: 7) and reverse primer: GTAGGACATACCCTCAGCATCATG-3’ (SEQ ID NO: 8), CBDAS-specific forward primer 5′-GAGGCTATGGACCATTGA (SEQ ID NO: 9) and reverse primer: 5′-GGACAGCAACCAGTCTAA-3′ (SEQ ID NO: 10), and CBCAS-specific forward primer 5′-CGGATGTACTGTTATGCTCCAA-3′ (SEQ ID NO: 11) and reverse primer: 5′-AAGCTTTCATGGTACCCCATGATGATGCCGTGGAAGAG-3′ (SEQ ID NO: 12). PCR parameters have not been previously reported for the co-dominant DNA marker (Onofri et al. 2015; Pacifico et al. 2006) and these were optimised as follows: each reaction contained 1.5 mM MgCl2, 0.2 mM dNTPs, 0.4 μM for the forward primer and 0.2 μM for THCAS-specific, CBDAS-specific, and CBCAS-specific reverse primers, and 2 U Platinum® Taq DNA Polymerase (Life Technologies #10966-034). Thermocycling parameters were 94° C. for 2 min, then 25 cycles of 94° C. for 30 s, 58° C. for 30 s, 72° C. for 1 min 15 s. PCR reactions were performed in 0.2 mL 96 well PCR plates (Thermo Scientific #AB-0600) sealed with flat cap strips (Thermo Scientific #AB-0786) using a Gradient Palm-Cycler™ (Corbett Life Science) and occurred in a total volume of 50 μL. D589 and B1080/B1192 amplification products were separated by electrophoresis on a 1.5 and 1% SeaKem® LE agarose gel (Cambrex #50004) stained with GelRed™ (Biotium #41003) respectively. Amplification products were then visualized under UV illumination using the Bio-Rad Molecular Imager® Gel Doc™ XR+ system using Image Lab™ software.

To confirm the expression of the transgene after agroinfiltration, a direct ELISA protocol was carried out. ELISA-extraction buffer (2% PVP, 0.03 MNa2SO3) was used to extract total proteins for both TCHAS, CBDAS, and CBCAS. ELISA 96-well plates (Thermo Fisher Scientific) were coated with 250 μl antigen and total soluble protein for both THCAS, CBDAS, and CBCAS followed by overnight incubation at 4° C. Plates were washed three times with washing buffer the next day, three times, 5 minutes each. By adding 250 μl blocking buffer (PBS-Tween 20, 5% low-fat milk), the remaining protein-binding sites were blocked and incubated for 2.5 hrs at room temperature.

After washing with PBS and Tween 20, anti-TCHAS, anti-CBDAS, anti-CBCAS antibodies were diluted by 1:1000 in a blocking buffer and 250 μl was added to each well. The plates were incubated in a humid chamber at 37.5° C. for 3.5 hrs. The plates were decanted and washed three times, 5 minutes each. 250 μl of substrate buffer (0.3 g (NaN3), 96 ml diethanolamine, 600 ml H₂O) was added to the plates followed by incubation at room temperature until the color developed. With an automated ELISA reader (BIOBASE 2000), absorbencies were finally read at 630 nm wavelength, 15 minutes each. ELISA values were expressed as the mean absorbance at wavelength (λ=640A°). Compared to negative control (C−), the S3, S5, S7 and S10 infiltrated samples showed positive results after 15 min and 30 min of read time. The highest expression level of THCAS, CBDAS, and CBCAS genes within Nicotiana benthamiana leaves were obtained at the fifth day post-infiltration followed by descending in expression level at 7th and 10th days post-infiltration.

The proteins were purified by immobilised metal affinity chelating chromatography (IMAC). To immobilise the metal ions on Chelating Sepharose Fast Flow, a solution of 200 mM NiSO4 (Sigma Aldrich) was passed through the column (GE Life Sciences). The column was washed with distilled water containing 0.02% azide to remove excess NiSO4. The column was then equilibrated with 10 column volumes of buffer ANiS (50 mM Tris-HCl, pH 7.4, 50 mM NaCl, 100 mM Imidazole, 10 mM β-Mercaptoethanol, 0.02% (w/v) Azide) with the flow rate of 3 ml/min. The crude extracts in buffer ANiS were applied to the column of Nickel Chelating Sepharose Fast Flow (column volume 10 ml) (GE Life Sciences). The column was washed with at least 10 column volumes of buffer ANiS, and was then switched to a linear gradient increasing the concentration of imidazole from 100 mM (buffer ANiS) to 500 mM of buffer BNiS (50 mM Tris-HCl, pH 7.4, 50 mM NaCl, 500 M Imidazole, 10 mM β-Mercaptoethanol, 0.02% (w/v) Azide.

To concentrate target proteins, ultrafiltration was used. The protein solutions were added to a stirred cell with a volume of either 10 ml or 50 ml (Amicon®, Millipore Sigma) and passed through an ultrafiltration cut disc regenerated cellulose membrane with a molecular weight cut off of 30 kDa (Ultracel®, Millipore Sigma). Using centrifugal filter devices (Microcon®, Millipore Sigma) according to the supplier's protocol, a concentration of small volumes of protein (300-600 μl) was achieved. To retain the target proteins, the exclusion limit of the membrane was selected.

To measure enzymatic activity of THCAS, CBDAS, and CBCAS, 150 mg of frozen plant material for each was homogenized in 500 ll of THCAS, CBDAS, and CBCAS reaction buffer (100 mM trisodium citrate, pH 5.5) and centrifuged (17,000×g, 15 min). Afterwards, the supernatants were incubated with CBGA (final concentration 0.05 mM, 1.9% (v/v) ACN) for 2 h at 37° C. To terminate the reactions, 275 μl of ice cold acetonitrile was added, followed by incubation on ice for 30 min. Finally, the supernatants were purified two times from solid particles by centrifugation (17,000×g, 30 min, 4° C.).

THCAS, CBDAS, and CBCAS assay extracts were analyzed by HPLC-MS using a Poroshell 120SB-C18 (3.0 9 150 mm, 2.7 μm) column. Detailed parameters of HPLC-MS analysis is described in the supplementary data. For confirmation of THCAS, CBDAS, and CBCAS mass spectra of compounds were juxtaposed with mass spectra of authentic standards and further confirmed by LC-ESI-MS/MS. Quantification of THCAS, CBDAS, and CBCAS was done by integration of peak areas of the UVchromatograms at 260 nm. The enzyme showed 137±14 fkat g_{FW}⁻¹ activity towards THCA production, while the activity towards CBDA was 132±11 fkat g_{FW}⁻¹ and the activity towards CBCA was 129±13 fkat g_{FW}⁻¹.

THCAS, CBDAS, and CBCAS were able to produce up to 2.11 g of THCA/kg leaf biomass, 2.03 g of CBDA/kg leaf biomass, and 1.48 g of CBCA/kg leaf biomass after CBGA feeding to the culture natant, demonstrating the capacity of transiently transformed Nicotiana benthamiana in the biosynthesis of (novel) cannabinoids with enhanced properties by the incorporation of tailoring enzymes. Furthermore, as aforementioned in the summary, the present invention enables the production of cannabinoid precursor enzymes on a continuous basis, week after week, which obviously is advantageous towards the long cultivation and harvesting times in traditional cultivation of Cannabis. These strategies will help to support the potential value of cannabinoids as pharmaceutical drugs.

1.-17. (canceled)

18. A nucleic acid molecule for transiently transforming a plant to produce Δ9-tetrahydrocannabinolic acid synthase, THCAS, and/or cannabidiolic acid synthase, CBDAS, and/or cannabichromenic acid synthase, CBCAS, the nucleic acid molecule corresponding to a nucleotide sequence comprising at least one of following:
i) a nucleotide sequence fragment encoding a polypeptide having at least 78% sequence identity to SEQ ID NO: 4 or comprising at least 15 contiguous nucleotides of the nucleotide sequence SEQ ID NO: 4; and/or
ii) a nucleotide sequence fragment encoding a polypeptide having at least 78% sequence identity to SEQ ID NO: 5 or comprising at least 15 contiguous nucleotides of the nucleotide sequence SEQ ID NO: 5; and/or
iii) a nucleotide sequence fragment encoding a polypeptide having at least 78% sequence identity to SEQ ID NO: 6 or comprising at least 15 contiguous nucleotides of the nucleotide sequence SEQ ID NO: 6;
said nucleotide sequence further comprising a KDEL or HDEL retrieval tag for targeting the nucleotide sequence to the endoplasmic reticulum.

19. The nucleic acid molecule of claim 18, wherein said nucleotide sequence further comprises a polyhistidine tag or other purification tag to facilitate purification.

20. The nucleic acid molecule of claim 18, wherein said nucleic acid molecule comprises at least one heterologous moiety and/or at least one linker and/or at least one signal sequence and/or at least one detection label.

21. The nucleic acid molecule of claim 20, wherein said nucleic acid molecule comprises said signal sequence corresponding to a PR-1a signal peptide, a pathogenesis-related protein 4 or a pathogenesis-related protein STH-2.

22. The nucleic acid molecule of claim 18, wherein said nucleic acid molecule corresponds to, or comprises, SEQ ID NO: 1 or SEQ ID NO:2 or SEQ ID NO:3.

23. A viral vector comprising the nucleic acid molecule of claim 18.

24. The viral vector of claim 23, wherein said viral vector further comprises a further nucleotide sequence for deglycosylation.

25. The viral vector of claim 24, wherein said further nucleotide sequence is codon-optimized for Nicotiana benthamiana or Nicotiana tabacum species.

26. A method for producing Δ9-tetrahydrocannabinolic acid synthase, THCAS, and/or cannabidiolic acid synthase, CBDAS, and/or cannabichromenic acid synthase, CBCAS, said method comprising:
transiently transforming a plant with a nucleic acid molecule in accordance with claim 18, and
extracting THCAS and/or CBDAS and/or CBCAS from plant biomass obtained from said transiently transformed plant.

27. The method of claim 26, wherein said plant is a Nicotiana benthamiana or Nicotiana tabacum plant.

28. The method of claim 26, further comprising filtrating and/or purifying the extracted THCAS and/or CBDAS and/or CBCAS.

29. The method of claim 28, wherein said filtrating and/or purifying comprises a chromatography process.

30. The method of claim 26, wherein said plant is also transiently transformed to co-express a deglycosylation sequence to obtain the expression of THCA and/or CBDAS and/or CBCAS without glycosylation.

31. The method of claim 26, comprising Introducing the nucleotide sequence, using the viral vector, into at least one Agrobacterium tumefaciens strain.

32. The method of claim 31, wherein said at least one Agrobacterium tumefaciens strain comprises a combination of a plurality of Agrobacterium tumefaciens strains comprising or consisting of GV3101, C58C1, and LBA4404 and wild-type strains A4, At06, At10, and At77.

33. A method for producing Δ9-tetrahydrocannabinolic acid (THCA), and/or cannabidiolic acid (CBDA), and/or cannabichromenic acid (CBCA), comprising a method in accordance with claim 26, and
converting the THCAS to THCA, and/or
the CBDAS to CBDA, and/or the CBCAS to CBCA, by cannabigerolic acid oxidocyclization without hydroxylation.

34. The method of claim 33, further comprising a decarboxylation performed on the obtained THCA, CBDA and/or CBCA to respectively yield THC, CBD and/or CBC.


SEQUENCE LISTING (12 sequences)

SEQ ID NO: 1 - DNA, 2436 bp, Artificial Sequence
  NUCLEOTIDE SEQUENCE OF PR-1A/THCAS/PNGASE-F/ER/ 6xHIS CONSTRUCT, CODON OPTIMIZED FOR NICOTIANA BENTHAMIANA
atgggattcgtacttttctctcagctccctagtttccttcttgtttcaactttacttttgtttctcgtgatttctcattcttgtcgggcaaataattctcatattcaagccacaatactttgttctaagaaggttggtctccaaattagaacacgaagtgggggacatgacgcagaaggaatgagttacattagtcaagtcccttttgtagtagttgatttgagaaatatgcatagcattaagattgatgtacatagtcaaacggcttgggtagaagctggggccactttaggtgaggtttactactggatcaatgagaaaaacgaaaatctttccttcccagggggttactgtcccactgtgggagtagggggacactttagtggaggcggctatggtgctcttatgcggaactacggacttgcagcagataatattattgatgctcatttggttaacgtcgatggaaaagttcttgacagaaagtctatgggcgaagatttattttgggctatccgagggggaggtggagagaactttgggattatcgcagcatggaaaattaaacttgtagccgtgccatcaaaatctacgatatttagcgtgaagaagaatatggagattcacggcttagttaagctttttaacaaatggcaaaacattgcatataaatacgataaagaccttgttctcatgactcatttcattacgaagaacattactgacaatcatggtaagaataaaacaaccgtccacggttactttagttctattttccatggcggcgtcgactccttagtcgatcttatgaataagagttttccagagctcggaattaaaaaaactgattgtaaggagttttcatggatagatacaactattttttacagtggtgtcgtgaatttcaatactgcaaatttcaagaaggaaatccttttggatcgatctgctggcaagaaaacagcttttagcatcaagttggattatgttaaaaaacctattccggaaacagctatggttaagattttggagaagttgtatgaagaggatgtgggtgccggaatgtacgttttgtacccatacggcggcataatggaggaaattagtgagtcagctataccttttccccatcgtgcaggtatcatgtatgaattgtggtatacggcatcttgggagaaacaggaagataatgagaagcatatcaattgggttagatcagtctacaatttcactactccttatgtgagtcagaacccacgtcttgcatacctcaactatcgagatcttgatttaggcaagacaaaccatgcttctcctaataattatactcaagctagaatctggggcgaaaaatacttcggcaagaattttaatagacttgtgaaggtaaaaactaaggtcgatccgaataatttttttaggaatgagcagtctatcccacctttaccaccacatcaccatgccccagctgacaacaccgttaatataaagacatttgataaggtgaagaatgcatttggagatggattgagccaaagtgcagagggtacttttacttttccagctgacgttaccactgttaaaacgattaagatgtttataaagaatgagtgcccaaataaaacttgtgacgaatgggaccgttatgctaacgtatacgtgaaaaataagactacgggggagtggtatgaaataggcaggtttatcaccccatactgggtcggtacagagaagcttccccggggactggagatagacgttacagacttcaaatcactgttgagcggcaatactgaattaaagatttacaccgagacctggttggctaagggaagagaatattctgttgattttgatattgtctacggtactcctgactataaatatagcgccgttgtcccagtcattcagtataacaagtctagtattgatggtgttccttatggcaaagcccatacacttggcttgaagaaaaacattcaattgcctaccaatacagaaaaagcttacttgaggaccactatatcaggctggggtcacgctaagccatatgatgctggttctagaggatgcgcagagtggtgttttcgaactcatacaatcgcaataaacaatgctaacacgtttcaacatcaacttggtgcacttggttgttccgcaaacccaattaataatcagtcaccagggaattgggcaccagatcgtgcaggatggtgtccaggaatggctgtccctacgcgaattgacgttcttaataatagtttgacaggatcaactttctcttatgagtataaattccagtcatggacaaataatggtactaatggcgacgctttttacgccatctcctctttcgttattgcaaaatccaatacaccgatttctgcacctgtggtcactaataaggatgagttgcatcaccatcatcaccattaa

SEQ ID NO: 2 - DNA, 2613 bp, Artificial Sequence
  NUCLEOTIDE SEQUENCE OF PR-1A/CBDAS/PNGASE-F/ER/ 6xHIS CONSTRUCT, CODON OPTIMIZED FOR NICOTIANA BENTHAMIANA
atgggattcgtacttttctctcagctccctagtttccttcttgtttcaactttacttttgtttctcgtgatttctcattcttgtcgggcaaatcctagggaaaatttccttaagtgttttagtcaatatatcccaaataatgctaccaatttgaagcttgtatacacacaaaataatccattgtatatgtcagtgttgaactctactattcataacttgaggtttactagcgacacaacgcctaagccgttggttattgtaacgccatcacacgttagccacatccaaggaaccattctttgttccaagaaagtcggactgcaaattaggacccgtagtggtggtcatgattcagagggtatgtcttacatttctcaggttccatttgtgattgttgatttgaggaatatgcgatcaatcaagatagatgttcactcacagactgcatgggtggaagctggggccacattgggcgaggtctactattgggttaatgaaaaaaacgagaatctttcattggctgctgggtactgtccaaccgtttgcgctggcggtcattttggtggcggaggctacggacccttgatgaggaattatggtttggctgcagataatattattgacgctcacttagttaatgtccatggaaaggtccttgatagaaagagcatgggtgaggaccttttttgggcccttcggggtggtggagctgagtcttttggcatcattgtcgcttggaaaattagattagtcgctgttccgaagtcaactatgttcagcgttaagaaaatcatggagattcacgagttggttaagttggtcaataaatggcagaacattgcttataaatatgataaagacttattgcttatgactcactttattactaggaatataacagataatcagggtaaaaacaaaacagccatccacacatacttcagcagcgtctttctcggaggggtagactcactcgtggatttgatgaacaaatcattccctgaacttggcattaagaagactgactgccgacagctctcatggattgatacgattattttctacagtggagttgtaaactatgatacggacaattttaacaaggaaattttattagataggtctgccggacaaaacggtgcatttaaaattaaacttgattatgttaaaaagcctattccagagagcgtgtttgttcaaattttggaaaagttgtatgaagaagatatcggtgcagggatgtacgctctttacccttacggtggtattatggacgaaattagtgagtcagctatcccattcccacatagagctggaatcttatatgaattatggtacatatgcagttgggagaagcaggaggataatgaaaagcaccttaattggattcggaacatctacaattttatgacaccatatgtttccaaaaacccgcggcttgcttacctgaactaccgagatttagatattggaatcaatgatccgaaaaatcctaacaattacacacaggcaaggatatggggcgagaagtacttcggtaaaaattttgacagattggttaaagtgaaaacactcgtggatccgaataatttcttccgtaatgagcaaagcatccctccccttccaaggcatcggcacgccccagctgacaacaccgttaatataaagacatttgataaggtgaagaatgcatttggagatggattgagccaaagtgcagagggtacttttacttttccagctgacgttaccactgttaaaacgattaagatgtttataaagaatgagtgcccaaataaaacttgtgacgaatgggaccgttatgctaacgtatacgtgaaaaataagactacgggggagtggtatgaaataggcaggtttatcaccccatactgggtcggtacagagaagcttccccggggactggagatagacgttacagacttcaaatcactgttgagcggcaatactgaattaaagatttacaccgagacctggttggctaagggaagagaatattctgttgattttgatattgtctacggtactcctgactataaatatagcgccgttgtcccagtcattcagtataacaagtctagtattgatggtgttccttatggcaaagcccatacacttggcttgaagaaaaacattcaattgcctaccaatacagaaaaagcttacttgaggaccactatatcaggctggggtcacgctaagccatatgatgctggttctagaggatgcgcagagtggtgttttcgaactcatacaatcgcaataaacaatgctaacacgtttcaacatcaacttggtgcacttggttgttccgcaaacccaattaataatcagtcaccagggaattgggcaccagatcgtgcaggatggtgtccaggaatggctgtccctacgcgaattgacgttcttaataatagtttgacaggatcaactttctcttatgagtataaattccagtcatggacaaataatggtactaatggcgacgctttttacgccatctcctctttcgttattgcaaaatccaatacaccgatttctgcacctgtggtcactaataaggatgagttgcatcaccatcatcaccattaa

SEQ ID NO: 3 - DNA, 2616 bp, Artificial Sequence
  NUCLEOTIDE SEQUENCE OF PR-1A/CBCAS/PNGASE-F/ER/ 6xHIS CONSTRUCT, CODON OPTIMIZED FOR NICOTIANA BENTHAMIANA
atgggattcgtacttttctctcagctccctagtttccttcttgtttcaactttacttttgtttctcgtgatttctcattcttgtcgggcaaatcctcaagaaaattttttgaagtgcttttcagaatacatcccaaataatccagctaaccctaaattcatttatacacagcatgatcagctttacatgagtgtactgaattctactatccaaaatcttcgttttacatcagacacaacccccaagccacttgttattgttactccatcaaatgtatctcacattcaagcatctatattgtgttccaaaaaggttggtctccagattaggacaaggtcaggcggacatgatgctgagggcctgtcttacatatcacaggtcccattcgcaatcgttgacctccgaaatatgcacactgtcaaagtcgatatacactcacagacagcatgggtggaagcaggtgctactctaggtgaagtttattactggatcaacgaaatgaatgaaaacttttcctttcctggaggatattgtccaactgtcggagtagggggtcatttcagtggaggaggatatggtgctttgatgagaaactacggtttggctgctgataatataattgatgcacatttagtgaatgtcgatggaaaagtacttgatagaaagagcatgggggaagatctattttgggccattcgtggaggaggaggcgagaatttcgggattattgcagcttgtaagattaaattggtcgtggtcccttccaaggctacaattttttctgtgaagaagaatatggaaattcacggtcttgttaagcttttcaataaatggcagaatatcgcttacaagtatgataaagaccttatgctcaccactcatttccgtactaggaatataacagataaccatggaaagaacaagaccactgtacatggatatttcagctctatctttttaggtggagttgatagccttgtcgatctcatgaataagagtttccctgagctcggaatcaaaaagactgactgtaaagaattgtcatggattgataccaccattttctacagcggggtcgttaactacaacactgctaattttaaaaaagagattcttttagatcgtagtgcaggcaaaaagaccgccttcagtataaagctcgattacgttaaaaagttgatccctgaaacagcaatggtgaagatattagaaaaactgtatgaagaggaagtgggagtcgggatgtatgtcctttatccttatgggggtatcatggacgagatatccgagtcagcaataccatttccccatcgtgctggcataatgtacgaactttggtatacagctacatgggaaaagcaggaagacaacgaaaagcacatcaactgggttcgttctgtttataactttactacaccctatgtttctcagaatccacgtctggcctatttaaattacagggatctagatttagggaaaacaaatcctgagagcccaaacaactacacacaagctcgaatttggggtgagaagtacttcggaaaaaattttaaccgtttagtgaaggtaaagactaaggcagatccaaacaattttttcaggaatgaacaaagcattcctccactaccacctagacatcatgccccagctgacaacaccgttaatataaagacatttgataaggtgaagaatgcatttggagatggattgagccaaagtgcagagggtacttttacttttccagctgacgttaccactgttaaaacgattaagatgtttataaagaatgagtgcccaaataaaacttgtgacgaatgggaccgttatgctaacgtatacgtgaaaaataagactacgggggagtggtatgaaataggcaggtttatcaccccatactgggtcggtacagagaagcttccccggggactggagatagacgttacagacttcaaatcactgttgagcggcaatactgaattaaagatttacaccgagacctggttggctaagggaagagaatattctgttgattttgatattgtctacggtactcctgactataaatatagcgccgttgtcccagtcattcagtataacaagtctagtattgatggtgttccttatggcaaagcccatacacttggcttgaagaaaaacattcaattgcctaccaatacagaaaaagcttacttgaggaccactatatcaggctggggtcacgctaagccatatgatgctggttctagaggatgcgcagagtggtgttttcgaactcatacaatcgcaataaacaatgctaacacgtttcaacatcaacttggtgcacttggttgttccgcaaacccaattaataatcagtcaccagggaattgggcaccagatcgtgcaggatggtgtccaggaatggctgtccctacgcgaattgacgttcttaataatagtttgacaggatcaactttctcttatgagtataaattccagtcatggacaaataatggtactaatggcgacgctttttacgccatctcctctttcgttattgcaaaatccaatacaccgatttctgcacctgtggtcactaataaggatgagttgcatcaccatcatcaccattaa

SEQ ID NO: 4 - DNA, 1551 bp, Artificial Sequence
  (THCAS) CODON OPTIMIZED FOR NICOTIANA BENTHAMIANA
aatccaagagagaattttctaaagtgcttttctaaacatatacccaataacgttgcaaacccaaagttagtgtatactcagcatgatcaactttacatgtcgatacttaattccactatacaaaatctgagatttatatctgatacaactccaaaacctcttgttattgtcactccatctaataattcacatatacaggctacaattctctgcagtaagaaggttggacttcaaatcagaactcgcagtggcggtcatgacgctgaaggaatgagttacatctcacaagtaccttttgtagtggttgatctaaggaacatgcattccatcaaaattgatgttcattctcagaccgcgtgggttgaagctggtgctacacttggggaagtgtactattggataaatgagaagaatgaaaatttatcatttcctgggggatattgtcccactgtgggagttggcggtcacttttcgggtgggggatatggcgcattgatgaggaattacggtctagccgctgacaatattattgacgcacacctcgtaaacgtagatggcaaggtgttggatagaaagtcaatgggtgaggacctattttgggctattaggggtggtgggggtgaaaactttggtattatcgcagcttggaaaattaagcttgtggcagtccctagtaaatctaccatattctccgtcaagaaaaatatggagatacatggattggttaagttatttaacaaatggcaaaacattgcctacaagtatgataaagatttggtcctgatgactcatttcataacaaagaacatcaccgataaccacggaaaaaataagacaacagtgcatgggtacttcagctccatctttcacggcggcgtcgattctttggtagatttgatgaataaaagcttccctgagctgggaataaagaaaactgattgtaaagagttcagctggattgatactacaatattttattcaggtgttgttaacttcaacacggcaaatttcaagaaggaaattttgcttgacagaagtgctgggaagaaaactgcattctctattaaattggattatgtgaaaaaacctattcccgaaaccgctatggtaaagattctggagaagctttatgaggaggatgttggtgctgggatgtatgttctttatccatatggaggcattatggaagagatttctgaatcagcaattccatttcctcatcgtgccggaattatgtacgaattgtggtatacggcatcatgggaaaaacaggaagataatgaaaagcatatcaattgggtccggtcagtttataattttacaaccccatatgtcagtcagaatccgcgattagcttatttaaactacagggacctcgacctgggtaaaacgaatcatgcgtctccgaacaactacactcaagcaagaatttggggagagaaatattttggaaagaatttcaatcgattagtgaaggtaaaaacaaaagttgatccaaataattttttccgtaacgaacaaagtatcccgcctctccctccacaccatcac

SEQ ID NO: 5 - DNA, 1548 bp, Artificial Sequence
  (CBDAS) CODON OPTIMIZED FOR NICOTIANA BENTHAMIANA
aatccgagagagaattttttgaaatgtttctctcaatatatccccaacaatgcaacaaatctgaaactggtttacactcagaataatccgttatacatgtctgttttgaactctacgatacataatcttaggttcacatctgacacaactccaaaacctctcgtgattgtgactcccagccatgtctcccacatccaaggcacgattctttgcagtaagaaggtaggattgcagataaggacacgttccggcggtcatgatagtgaaggtatgtcctacatcagccaagttccatttgttattgttgaccttagaaacatgcggagtattaaaattgatgttcattcacagaccgcatgggtagaagctggagcaactctgggggaggtatactattgggtgaatgaaaaaaatgaaaatttaagtcttgctgcaggctattgtcctactgtttgtgctggtgggcattttgggggggggggatatggacctctaatgcgaaactatgggcttgcagccgataacatcattgatgctcatcttgtcaatgtgcatggaaaagtactggatcggaagtcaatgggagaggatctattttgggccctccgaggtggtggtgctgaaagttttggaattatagttgcttggaaaatacgcctcgtggcagtccccaagagcaccatgttctcagtcaagaagattatggaaattcatgaattggtgaagttggtaaacaaatggcaaaacattgcttacaagtatgataaagatcttttgcttatgactcacttcatcaccaggaatataactgataatcagggaaagaacaaaactgccattcacacatatttttcatcggtcttcttgggaggtgttgactctcttgttgatttgatgaataagagttttcctgagctgggtattaagaaaacagattgtaggcagctctcatggatagataccattatattttattctggagttgtaaattatgacacagacaatttcaataaagagattttactcgacagatcagctggccaaaatggtgcatttaagatcaaacttgattatgttaaaaagccaattccagaatcggtgtttgtccagattctcgagaagctatatgaagaagacattggtgcgggaatgtatgctttgtatccttatggtgggatcatggatgaaatatctgagtcagctattccttttcctcacagagccggcatattatatgagctttggtatatttgctcttgggaaaaacaagaggataacgagaaacatttaaattggattaggaacatatacaatttcatgacaccatatgtgtcaaagaatcctcgtctggcatatttgaattacagagatttggacattggaatcaatgatcctaaaaacccaaacaactacacacaagcgcgcatatggggcgaaaagtactttggtaagaattttgataggctggtgaaagttaaaactttagtagatccaaataacttctttagaaatgagcaatccatcccaccacttcctagacatagacac

SEQ ID NO: 6 - DNA, 1551 bp, Artificial Sequence
  (CBCAS) CODON OPTIMIZED FOR NICOTIANA BENTHAMIANA
aaccctcaagaaaatttccttaaatgcttttctgaatacattccaaacaatcctgctaatcccaaatttatttatactcaacatgaccagctgtacatgtcagtattgaattccacaattcaaaatctaagatttacctctgatactactccaaagccccttgtgatagtcactccttcaaatgtttcacacattcaggcatctatactttgttcaaagaaggtaggactccaaatcaggacaaggagtggaggtcatgatgctgagggattatcttatattagtcaagtgccatttgctatagttgacttgagaaatatgcatacagttaaggtcgacattcattctcaaacagcctgggttgaagctggagctactttaggcgaagtttattattggattaacgaaatgaatgagaatttctcattccccggtggatactgtccaactgtgggagttggtgggcatttcagcgggggtggctatggagccctgatgagaaactacggcctagcagcagacaatattattgatgcacacctcgtcaatgtggatgggaaagtacttgataggaaaagtatgggagaggatcttttttgggcgataagaggagggggtggtgagaattttggtataattgctgcttgtaaaattaaattggtagtagttcctagtaaagcaaccattttctcggtgaaaaagaacatggaaatccacggcttagtcaaacttttcaacaaatggcaaaacattgcctacaaatatgataaggatttaatgcttacaactcattttcggacacgaaacataaccgataaccatggtaagaataagaccactgttcacggatattttagctcgatcttcttgggtggcgttgactccctagttgatcttatgaacaagtcttttccagagcttggaatcaagaagacagattgcaaagaactgtcttggattgatacgacgatattttattcaggcgttgtgaactataatacagcaaatttcaagaaagagattctccttgaccgttccgcggggaaaaaaactgctttttcaatcaagctagattatgttaagaaattaatccctgaaaccgcaatggtgaagatactcgagaagttgtacgaggaagaggtcggtgttgggatgtacgtcctgtatccttatggtggcattatggatgagatctccgaatctgccatacctttcccacatagagctggcattatgtatgaactatggtatacggcaacttgggaaaagcaagaagataatgaaaagcatataaattgggtgaggtcagtttacaactttaccacaccatatgtaagtcagaatccgcgattggcttatttgaattatagggatttagacttgggaaaaactaatccggaaagccctaataattacacacaggcacgtatttggggtgaaaaatactttggaaaaaattttaatagattggtaaaggttaaaacaaaggctgatccaaataacttctttcgcaatgagcagagtatcccacctttacctccaagacatcac

SEQ ID NO: 7 - DNA, 22 bp, Artificial Sequence
  THCAS-specific forward primer
ctcgtatacactcaacacgacc

SEQ ID NO: 8 - DNA, 24 bp, Artificial Sequence
  THCAS-specific reverse primer
gtaggacataccctcagcatcatg

SEQ ID NO: 9 - DNA, 18 bp, Artificial Sequence
  CBDAS-specific forward primer
gaggctatggaccattga

SEQ ID NO: 10 - DNA, 18 bp, Artificial Sequence
  CBDAS-specific reverse primer
ggacagcaaccagtctaa

SEQ ID NO: 11 - DNA, 22 bp, Artificial Sequence
  CBCAS-specific forward primer
cggatgtactgttatgctccaa

SEQ ID NO: 12 - DNA, 38 bp, Artificial Sequence
  CBCAS-specific reverse primer
aagctttcatggtaccccatgatgatgccgtggaagag